(19) United States
(12) Patent Application Publication
(10) Pub. No.: US 2014/0303486 A1
(43) Pub. Date: Oct. 9, 2014

(54) Surgical Navigation Planning System and Associated Methods

(71) Applicant: Adventist Health System/Sunbelt, Inc., Altamonte Springs, FL (US)

(72) Inventors: James Edmund Baumgartner, Winter Park, FL (US); Po Ching Chen, Maitland, FL (US); Ki Hyeong Lee, Orlando, FL (US)

(73) Assignee: ADVENTIST HEALTH SYSTEM/SUNBELT, INC., Altamonte Springs, FL (US)

(21) Appl. No.: 14/200,420

(22) Filed: Mar. 7, 2014

Related U.S. Application Data
(60) Provisional application No. 61/774,259, filed on Mar. 7, 2013.
(60) Provisional application No. 61/833,576, filed on Jun. 11, 2013.

(51) Int. Cl. A61B 19/00; A61B 5/055

(52) U.S. Cl. CPC A61B 19/5244; A61B 5/055; A61B 19/54; A61B 2019/5454; A61B 2019/5236
USPC 600/414

(57) ABSTRACT

The present disclosure provides a surgical planning and navigation system imports multiple medical imaging sources of the same body part for integrated display in the same coordinate. Images are converted into 3D volumes, spatially co-registering each image volume to a base anatomical image.

### CROSS-REFERENCE TO RELATED APPLICATIONS

This application claims priority to provisional U.S. Patent Application Ser. No. 61/774,259, filed Mar. 7, 2013, and U.S. Patent Application Ser. No. 61/833,576, filed Jun. 11, 2013, the disclosures of which are herein incorporated by reference in their entirety, and commonly owned.

### FIELD OF THE INVENTION

The present invention generally relates to surgical planning and surgical navigation. More particularly, it is to the enhancement of 2 dimensional (2D) display and 3 dimensional (3D) display that require integration of multiple image sources of the same body part. The image sources may include raw images from various medical imaging modalities and their processed images.

### BACKGROUND

Different medical imaging modalities provide different aspects about the condition of a body part. In general, they describe anatomical, functional, physiological, or pathological information of the tissue. Integration of multi-modal information into a single display platform is desirable in surgical planning and navigation. This is especially useful for complicated brain surgery where a morbid area is surrounded by important functional areas.

Typically, trajectories for surgical intervention (including resection, biopsies, implantation and treatment delivery, etc.) are planned based on target selection and on the extent of the intervention through the guidance from medical images. To perform a safe and successful invasive surgical procedure, the physician plans the trajectory in consideration of the locations of the target and critical functional areas. To avoid the risk of injuring a critical area, surgical intervention needs be modified for saving critical functions.

Additionally, due to different levels of experience of the executing physician and/or the lack of knowledge of the patient-specific tissue configuration, as well as due to patient-specific variations in the arrangement of the tissue, for example in the case of a diseased tissue, it has been necessary to leave sufficient space between critical areas (in terms of a specific level of risk, automatically and/or manually defined) in order to ensure that surgical intervention does not interfere with the critical area. These areas include, for example, major vessels, ventricles, eloquent cortex and limbic system in the brain. Therefore, integration of comprehensive multi-modal medical images acquired prior, during and after surgical intervention has become critical. It can identify the relative locations between morbid tissue and functional tissue for clinical diagnosis and pre-surgical planning. It can also be utilized to monitor the progress of the surgery through instant comparing pre-operative, intra-operative and post-operative images.

However, the characteristic nature differs among modalities and the results are generated separately from different sources with or without further advanced imaging processing. This situation makes the source images having very different configurations with significant lack of proper integration. This greatly reduces their values for clinical diagnosis and hinders the efficiency of clinical practice. Thus there exists a need for a single system that can promptly display and manipulate various data that are provided by different modalities for more accurate, and therefore successful, surgical planning and navigation.

### SUMMARY OF THE INVENTION

The present application provides an integrated system and process that addresses the above described problems and provide a desirable solution for clinical application.

The present invention generally relates to surgical planning and surgical navigation. More particularly, it is to the enhancement of 2 dimensional (2D) display and 3 dimensional (3D) display that require integration of multiple image sources of the same body part. The image sources may include raw images from various medical imaging modalities and their processed images. With enhanced display capability that combines anatomical, functional, physiological, pathological information in the same coordinate, a user can easily review all the results in a single screen setup for clinical diagnosis. A surgical intervention plan can be orchestrated in the system based on integrated display. The resulting images and surgical plane can be further output in to a surgical planning and navigation system for assisting surgical intervention.

The application provides a surgical planning and navigation system comprising:

a) a computer capable of displaying various informational images related to any specific voxel in a body part of a patient for clinical diagnosis and surgical navigation;

b) optional stationary MRI compatible fiducial markers attached to the rigid and unaffected tissue outside surgical intervention of said patient, wherein the fiducial markers enable the computer to co-register the various informational images related to any specific voxel in the same body part from the images acquired prior, during, and after surgical intervention promptly without considering defects produced by surgical intervention;

c) a probe that is connected to the computer such that the computer can navigate the probe to a specific voxel; and

d) one or more electrodes, catheters, optical fiber or other investigational or treatment devices directed via the probe to a targeted voxel(s) in the tissue, wherein investigational or treatment devices are capable of relaying real time functional information related to the targeted voxel(s) in the present navigation system.

The application provides a computer-implemented surgical navigation planning method comprising:

a) providing a plurality of informational images associated with the same body part of a patient;

b) pre-processing various input imaging sources by converting them into each 3D image volumes with assigned attribute settings in a uniform coordinate system;

c) aligning all input image volumes to a base image volume through spatial co-registration procedure;

d) processing an image volume if needed through segmentation or creating activation map for highlighting significant regions thereof developing various aspects of each image volume;

e) treating each 3D image volume as an individual module with attribute settings. The integrated display is implemented by superimposing them according to their attribute settings and rending them in 2D or 3D views;

f) allowing the integrated display manipulation by adjusting the attribute settings;

g) planning a surgery based on the integrated display by creating new image volumes or modifying existing image volumes for displaying a trajectory of a planned resection margin or showing physiological/pathological properties of a location;

h) outputting integrated information including surgical plan into a surgical planning and navigation system or files; and

i) simplifying display setting for surgical planning and navigation system by using only one of attributes for promptly switching desired display during operation.

The application provides a method for planning the placement of an investigational or treatment device via the probe to a specific targeted voxel(s) in the tissue, comprising:

a) analyzing information of at least part of the tissue of the patient to determine if at least one specific or critical region or structure lies within a region of interest, said region of interest within a predetermined distance of a planned trajectory of the device in the tissue; and

b) using a processor to assess a level of risk to the at least one specific or critical region or structure that is within the region of interest, wherein levels of risk are determined based on risk due to at least one of patient-specific physiological characteristics of the specific region or structure of the tissue, and wherein the levels of risk are determined based on at least one of:
i) risk due to anatomical, functional and/or physiological characteristics of the specific region or structure of the brain, including risk of harming the tissue including the risk of crossing the trajectory of the device in the tissue with a critical region for tissue functioning, and/or risk of unsuccessful treatment including the risk of removal of critical tissue;
ii) risk due to a predefined treatment plan, said risk due to the predefined treatment plan including a proximity of a treatment approach or trajectory to a risk structure, the proximity of two or more different treatment approaches or trajectories to each other, and/or weighting the risk of the predefined treatment approach; or
iii) risk due to a predefined setup or configuration of a surgical planning and navigation system; and
iv) displaying borders along or around the specific or critical region or structure, wherein when planning the trajectory to cross at least one border, a warning signal is output or the trajectory is prevented from crossing the border.

### BRIEF DESCRIPTION OF THE DRAWINGS

Embodiments of the invention are described by way of example with reference to the accompanying drawings in which:

FIG. 1 is a functional block diagram illustrating one method embodiment of the present invention described herein by way of example for a computer-implemented surgical navigation planning system.

FIG. 2 is a functional block diagram illustrating the detailed stages of Step 1 Image pre-processing procedure in FIG. 1.

FIG. 3 is a functional block diagram illustrating the detailed stages of Step 2 Image co-registration procedures in FIG. 1.

FIG. 4 illustrates the layer structure of the proposed layer attribute.

FIG. 5A is a head MRI image volume in Step 2.

FIG. 5B is the brain image volume in Step 3 segmented from head MRI image volume in FIG. 5A.

FIG. 5C is a head SPECT image volume in Step 2.

FIG. 5D is the hyper-profusion map image volume in Step 3 processed from head SPECT image volume in FIG. 5C.

FIG. 5E is a head fMRI image volume in Step 2.

FIG. 5F is the hand motor map image volume in Step processed from head fMRI image volume in FIG. 5E.

FIG. 5G is a head CT image volume in Step 2 and FIG. 5I is the subdural electrode image volume in Step 3 segmented from head CT image volume in FIG. 5G.

FIG. 6A is the resulting product of Step 4 in 3D display rendering.

FIG. 6B is resulting product of Step 4 in 2D transverse view.

FIG. 6C is the resulting product of Step 4 in 2D sagittal view.

FIG. 6D is the resulting product of Step 4 in 2D coronal view.

FIG. 7A is resulting product of Step 5 in 3D display rendering.

FIG. 7B is the resulting product of Step 5 in 2D transverse view.

FIG. 7C is the resulting product of Step 5 in 2D sagittal view.

FIG. 7D is the resulting product of Step 5 in 2D coronal view.

FIG. 8 shows Table, which lists the attributes of each image volume including but not limited to layer, name, group, 2D display, 3D display, transparency, color scale, contour, threshold, data type and comment, as desired by way of example.

FIG. 9 shows Table 2, which lists the simplified display interface in Step 6 for surgical planning and navigation system.

### DETAILED DESCRIPTION

The present invention will now be described more fully hereinafter with reference to the accompanying drawings, in which embodiments of the invention for a computer-implemented surgical navigation planning system are shown by way of illustration and example. This invention may, however, be embodied in many forms and should not be construed as limited to the embodiments set forth herein. Rather, these embodiments are provided so that this disclosure will be thorough and complete, and will fully convey the scope of the invention to those skilled in the art. Like numerals refer to like elements.

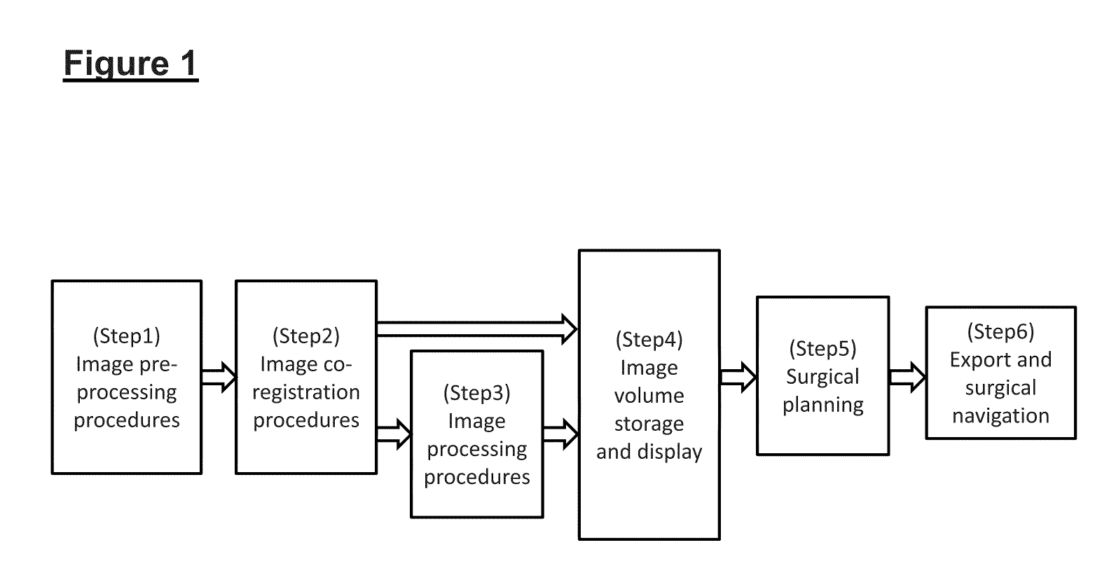

With reference initially to FIG. 1, one embodiment of the present invention for a computer-implemented surgical navigation planning system of is herein described by way of example. For the embodiment herein described by way of example, an initial or first step comprises gathering medical images (e.g. neuro images) from different modalities. The modalities may include Magnetic Resonance Imaging (MRI); Functional Magnetic Resonance Imaging (fMRI); Diffusion Tensor Imaging (DTI); Positron Emission Tomography (PET); Single Photon Emission Tomography (SPECT); X ray Computed Tomography (CT); Magneto-encephalography (MEG); and Intra-cranial encephalography (iEEG), contrast enhanced venogram tomography, by way of non-limiting example.

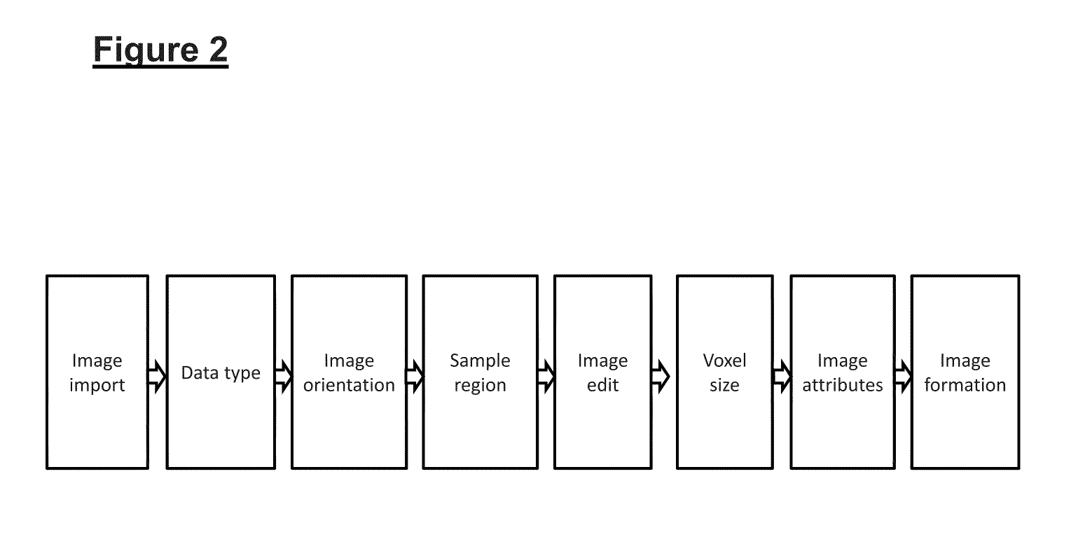

(Step 1) Pre-processing procedure is illustrated in FIG. 2 through multiple stages. In the stage of Image import, it starts with importing images from various 2D image sequence formats (e.g. DICOM (Digital Imaging and Communications in Medicine), JPEG, GIF, BMP . . . etc.) or 3D image format (e.g. nifi (Neuroimaging Informatics Technology Initiative), Soildwork, Auto CAD, Adobe . . . etc.). A program automatically reads the embedded information (e.g. data type, image orientation, pixel/voxel size . . . etc.) inside the image files for 3D image volume construction in the present surgical navigation planning system. If the required information for 3D image volume construction cannot be found in the image files, a user will be asked to specify them manually in the later steps. A user may also verify the automatic readouts in the later steps.

In the stage of Data type, the input image data type and output data type to the system are indicated. A user can fill out the blank or change the input type if the automatic readout was not correct. For output data type to the system, the proposed system allows the information saved in a 3D voxel cell to be versatile where its format may be either numeric data, text data, numeric array, text array, or links to multimedia, as desired.

In the stage of Image orientation, the orientation (left, right, anterior, posterior, superior and inferior) of the input image is indicated in a 2D (coronal view, transverse view and sagittal view) and 3D view. A user can verify the orientation or modify the orientation if the automatic readout was incorrect.

In the stage Sample region, the X, Y, Z boundaries of the input image is indicated in a 2D (coronal view, transverse view and sagittal view) and 3D view. A user can reduce or extend the sample region by sliding the boundaries in 2D or 3D display or by typing in value. Only the voxels inside the sample region boundaries are output to the present system.

In the stage of Voxel size, the X, Y, Z dimension of the voxel in the input image and corresponding memory usage are indicated, a user can verify its correctness and change it if the automatic readout was wrong or as needed.

In the stage of Image edit, a user can edit the image in 2D and 3D display. Its purpose is to remove unwanted artifacts in the image or repaired damage image sections.

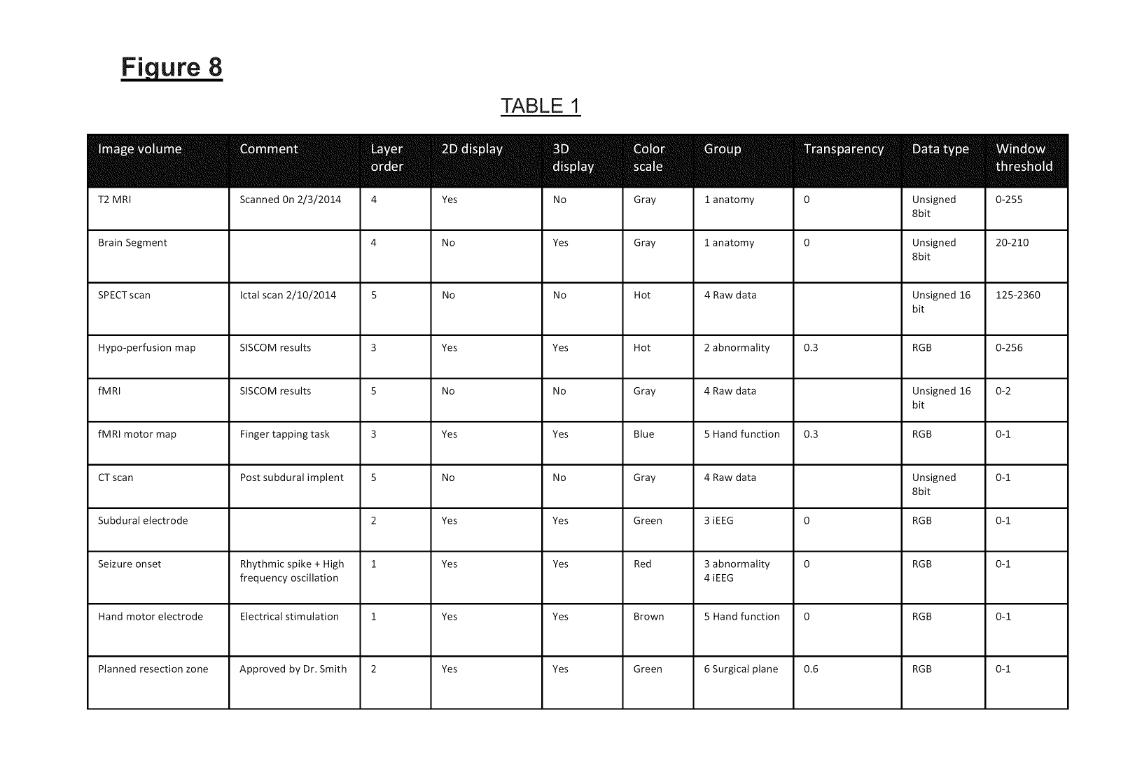

In the stage of Image attributes, the original images are converted into the proposed 3D image volume which is considered as an individual module. More importantly, each image volume has its own attributes such as layer order, name, group, display, transparency, color scale, contour, window threshold, data type . . . etc. as desired by way of example in TABLE 1 in FIG. 8. A user can complete the attributes as much as information as needed in this stage or complete them later in the Step 4 Image volume storage and display procedure.

### DEFINITIONS

Below are definitions of some major attributes of image volume as used in the proposed system.

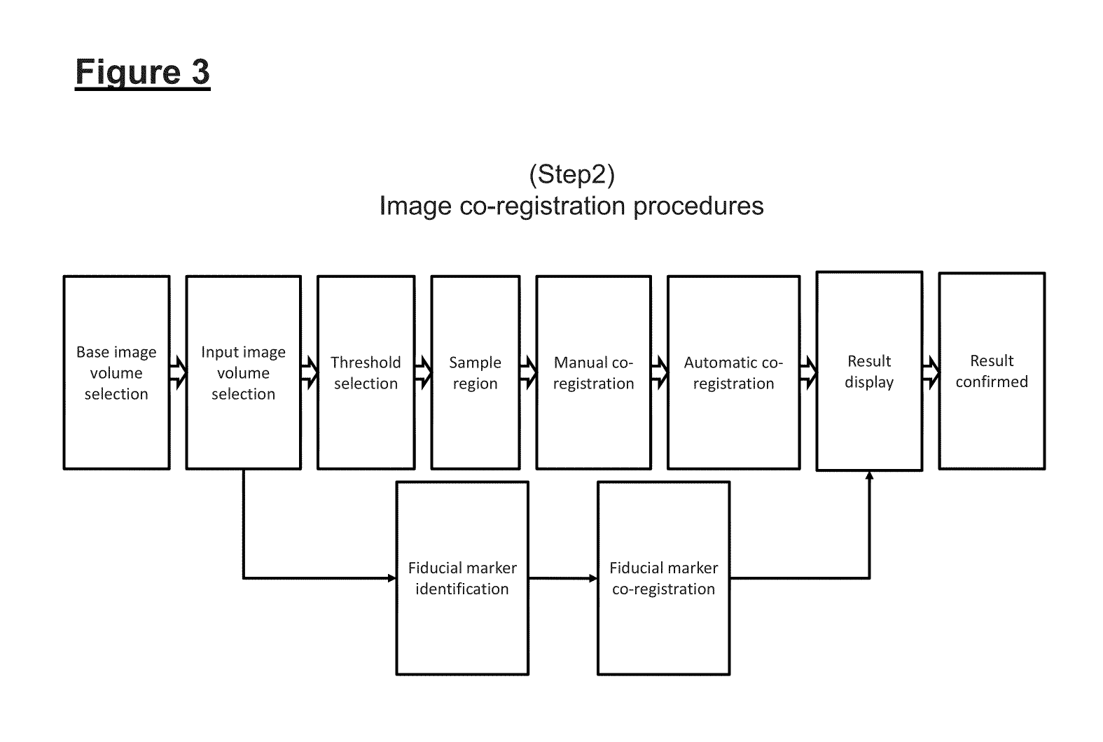

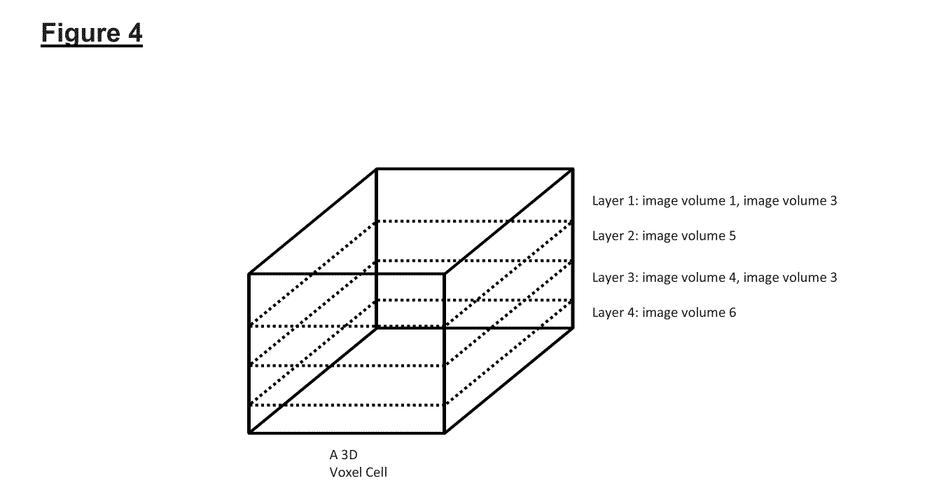

As used herein, the term “Layer” as an attribute means the organization of the 2D and 3D displays of multiple image volumes that overlap each other in a uniform coordinate system. The layer structure is illustrated in FIG. 3. A 3D voxel in a specific location can breakdown to one to multiple layers. An image volume can reside in one or more layers. If the image volumes were in the same layer, they are fused into a single image volume for display. The Layer 1 (top) has highest display priority; Layer 2 has second priority; Layer 3 has third priority, and so on. Lower layers can be seen only if there is transparency in the upper layers.

As used herein, the term “Proportion” as an attribute means to define the contribution from different image volumes fused in single volume in the layer.

As used herein, the term “Transparency” as an attribute means to define the transparency of each layer.

As used herein, the phrase “Window threshold” as an attribute means to define the range of intensity in each image volume to will be displayed. If the intensity is outside the threshold, it will be seen as transparent. A transparent voxel will not contribute to the fusion of volumes in the same layer nor block display between layers.

As used herein, the phrase “Color scale” as an attribute means to set the color scale used for display.

As used herein, the term “Group” as an attribute means to put different image volumes in different layers in to one display group. This can help with rapid display switch among different group settings.

As used herein, the term “Comment” as an attribute means to give a text comment about the image volume, which can be shown in a 2D or 3D display.

As used herein, the phrase “2D display” as an attribute means to set an image volume to be shown in 2D display (coronal plane, transverse plane and sagittal plane) or not.

As used herein, the phrase “3D display” as an attribute means to set an image volume to be shown in 3D display or not.

In the stage of data formation, the final product of Step 1 is displayed in 2D (coronal view, transverse view and sagittal view) and 3D for confirmation. All image volumes are converted to a uniform coordinate system based on their voxel sizes. A 2D image sequence is then stacked into a 3D image volume. Information in a 2D pixel cell is translated into a 3D voxel cell accordingly based on its slice location and pixel matrix size. Pre-processing procedure may also include well known methods including improving quality of the image data, removing noise or artifacts from the image data, and the like. Pre-processing steps can include identifying anatomical regions of interest, such as targets in the brain or any portion of the anatomy.

(Step 2) Image co-registration procedure is illustrated in FIG. 3 in multiple stages. The procedure is to align all image volumes to a base anatomical image volume through spatial co-registration.

The first stage is to select a base image volume, by way of non-limiting example; the base anatomic image volume may come from a high resolution T1/T2 volumetric isotropic MRI sequence. The base image volume will remain still during the co-registration procedure. The voxel of the input image volume is resampled to match the voxel size of base image volume

The second stage is to select the input image volume which needs to be aligned with the base image.

The third stage is to select the intensity threshold of base image and input image for co-registration matching. Only the voxel that contains the intensity within the threshold will be used for co-registration matching.

The fourth stage is to select the spatial boundaries of base image and input image for co-registration matching. Only the voxel within the spatial boundaries will be used for co-registration matching. This is especially useful for matching pre-operative images with post-operative images. Defects produced by surgical intervention need to be ruled out in co-registration process to reduce errors.

The fifth stage is to roughly align two image volumes by manually rotating and translating images in 2D display (coronal view, transverse view and sagittal view).

The sixth step is to align two image volumes through a automatic imaging co-registration algorithm which tries to find the best match between the input image volume and the base image volume by analysis of minimum errors while adjusting the input image volume through using full 12 degree of freedom full affine transformation (including translation, scale, sheer and rotation).

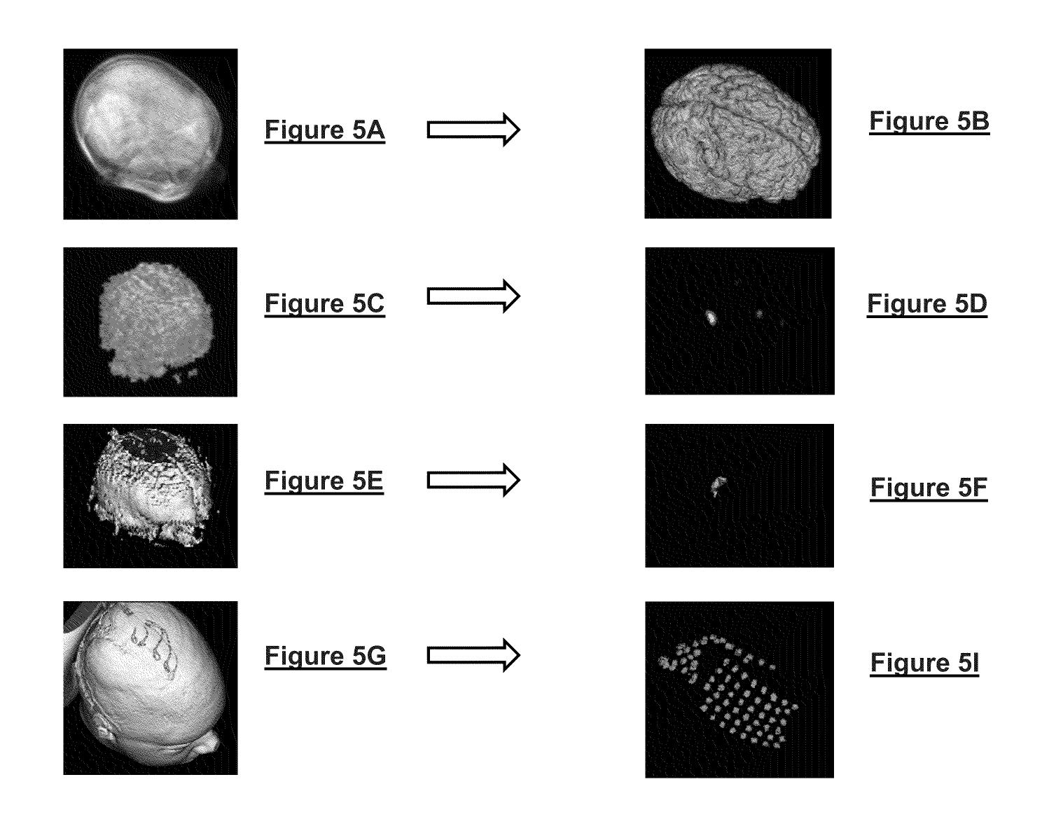

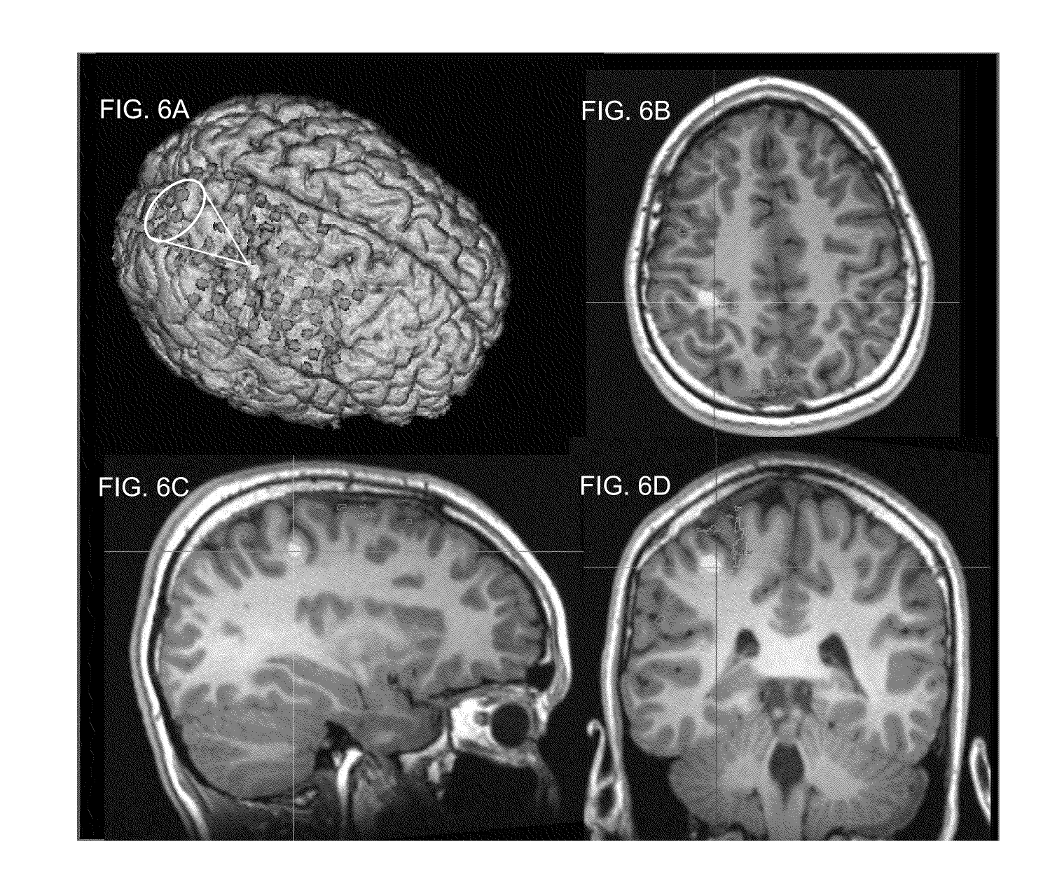

The seventh stage is to display the co-registration result in 2D (coronal view, transverse view and sagittal view). A user can go back to any previous stage to readjust settings in previous stage to achieve better results. All previous stages can be skipped if the co-registration has been done in advance before import. If the results are satisfactory, a user can confirm the results and move on to next step. The resulting products of (Step 2) are illustrated in FIG. 5A, FIG. 5C, FIG. 5E and FIG. 5G.

If the fiducial makers are available on both the base image volume and the selected input image volume, the Step 2 Image co-registration procedure can go the other route to align two image volumes as depicted in FIG. 3. The first stage is to select a base image volume. The second stage is to select if the image volume needs to be aligned with the base image. The third stage is to identify and match fiducial makers in both image volumes. This can be implemented in both 2D and 3D views. The fourth stage is to co-register fiducial markers in both image volumes automatically using an algorithm through 12 degrees of freedom full affine transformation. The fifth step is to review the co-registration results in 2D display. A user can confirm the results or go back to the previous stage or choose to use the other route of co-registration through conventional image matching.

(Step 3) Image processing procedures are herein described as including two categories. First category creates a segmentation of an image volume from Step 2, and a second category creates an activation map of the image volume from Step 2. Segmentation of an image volume may include segmenting out brain tissue images from a head MRI scan as illustrated with reference to FIG. 5B; segmenting out a tumor or lesion images from a MRI scan; and segmenting out iEEG electrode from a post subdural electrode implantation CT scan as illustrated with reference to FIG. 5I, by way of example. The segmentation can be implemented by using manual, semi-automatic, full automatic segmentation process. For the manual segmentation, the procedure allows a user to define boundary of a segment through various hand drawing tools in 2D or 3D display. For semi-automatic segmentation, a user can place a seed in the region of interest. Through adjusting intensity threshold of the image volume and edge enhancement, the program will grow the segment from the seed and determine its boundary automatically. For full automatic segmentation process, a user selects a specific named target. The program will perform segmentation automatically based on the selection. The segmentation procedure can be done interchangeably to achieve the desired results.

A second category is herein described as creating a activation map of image volumes from Step 2 using statistical, analytical or clinical methods. By way of example, a hypo-metabolism/hyper-metabolism map is created from a brain PET. A motor function map is created from an fMRI scan as illustrated with reference to FIG. 5F. A language function map is created from iEEG stimulation exam, and a hyper-perfusion map is calculated from a brain SPECT scan as illustrated with reference to FIG. 5D. A venous map is acquired from contrast enhanced MRI or CT scan. A corticospinal tract is created from a brain DTI scan. This category can be treated an additional module of the system which can be incorporated as needed when special image processing procedure is required.

In Step 4, Image volume storage and display, all the image volumes (with/without additional image processing) should be stored and organized according the their attributes for display. By way of example, image volumes from Step 2 and Step 3 of a patient are illustrated in FIG. 5. By way of example, the attributes for each image volumes are summarized in Table 1 in FIG. 8. In a 2D and 3D display, all image volumes are superimposed onto each other according to their attribute settings listed in Table I are illustrated in FIG. 8. A user can change display through a graphic user interface to modify the attribute settings to achieve desired 2D or 3D display.

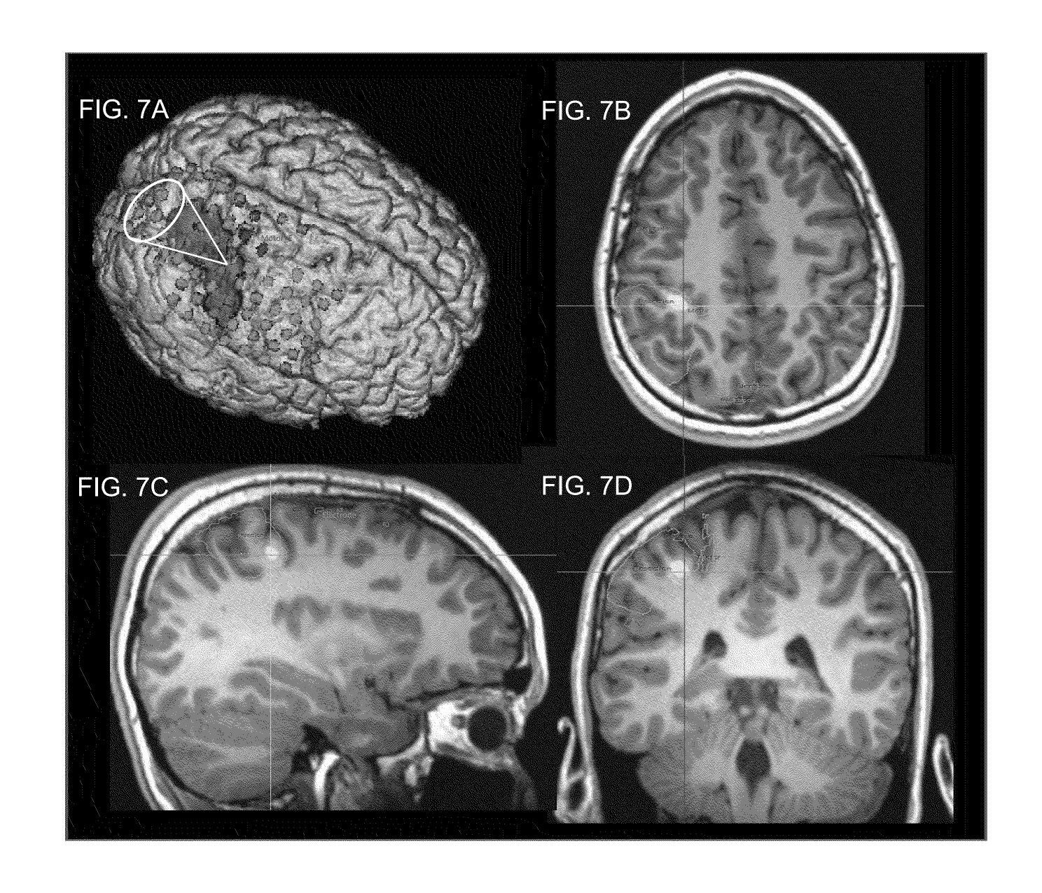

(Step 5) Surgical planning is implemented based on the integrated display in Step 4 by creating new image volumes or modifying existing image volumes for indicating the clinical diagnosis and surgical plane. The creation of new image volumes for surgical planning uses the segmentation procedure which is described earlier in Step 3. A user can create a new image volume by clicking an existing image volume in 2D or 3D view for seeding and the semi-automatic segmentation procedure is able to create a new image volume. By way of example, a user can select on a subdural electrode from a subdural electrode grid to create a new image volume for assigning special physiological/pathological information to the subdural electrode. For examples, a new image volume of subdural electrodes can be used to describe the location of epileptic seizure onset as illustrated as magenta electrodes in FIG. 7 in 3D and magenta contours in FIG. 7 in 2D. The manual segmentation procedure allows a user to define the boundaries of a new image volume which describes the planned resection zone on an existing image volume (brain segment in the example) The new image volume which indicates planned resection zone is illustrated as a cyan outline in FIG. 7 in 3D display and cyan contour in FIG. 7 in 2D display.

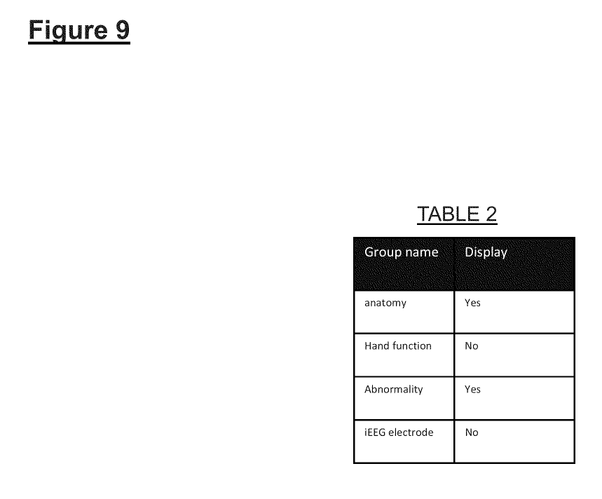

As a next step, herein referred to as Step 6, the output and surgical planning and navigation system, the resulting integrated image display after planning in Step 5 can be exported into various 2D or 3D file formats for displaying in different software systems or into a surgical planning and navigation system. The complicated attribute settings during Step 4 and (Step 5) are not convenient to modify for rapid display switch which is usually required in the operating room setting. In order to simply the display setting for surgical navigation, only one attribute is used (such as group attribute in this example) for switching on and off different sets of image volumes in surgical planning and navigation system during operation. By way of example, TABLE 2 in FIG. 9 describes the simplified display setting for surgical planning and navigation system using group attribute. When the group attribute is setup properly, a user can easily obtain a simple clear view of only desired information without going through complicated setups. The white cone in FIG. 7A illustrates a probe position identified by the surgical planning and navigation system in a 3D rendering. Cross-lines illustrated with reference to FIGS. 7B, 7C and 7D, illustrate a corresponding 2D location of the probe.

Flowcharts and block diagrams herein described illustrate architecture, functionality, and operation of possible implementations of systems, methods and computer program products according to various embodiments. Therefore, it will be understood by those of skill in the art that each block in a flowchart or block diagram may represent a module, segment, or portion of code, which comprises one or more executable computer program instructions for implementing the specified logical function or functions. Further, some implementations may include the functions in blocks occurring out of the order herein presented. By way of non-limiting example, two blocks shown in succession may be executed substantially concurrently, or the blocks may at times be executed in the reverse order, depending upon the functionality involved. It will also be noted that each block of the block diagrams and flowcharts, and combinations of blocks in the block diagram and flowchart illustrations, may be implemented by special purpose hardware-based systems that perform the specified functions or acts, or combinations of special purpose hardware and computer instructions.

These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer program instructions may also be stored in a computer readable medium that may direct a computer, other programmable data processing apparatus, or other devices to function in a particular manner, such that the instructions stored in the computer readable medium produce an article of manufacture including instructions which implement the function/act specified in the flowchart and/or block diagram block or blocks. The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Aspects of various embodiments may be embodied as a system, method or computer program product, such as a system as herein described by way of example, and accordingly may take the form of an entirely hardware embodiment, an entirely software embodiment (including firmware, resident software, micro-code, and the like) or an embodiment combining software and hardware aspects that may all generally be referred to herein as a circuit, module or system. Furthermore, aspects of various embodiments may take the form of a computer program product embodied in one or more computer readable media having computer readable program code embodied thereon. It is understood that the computer implemented method herein described operates with readable media relating to non-transitory media, wherein the non-transitory computer-readable media comprise all computer-readable media, with the sole exception being a transitory, propagating signal.

Any combination of one or more computer readable media may be utilized. A computer readable medium may be a computer readable signal medium or a computer readable storage medium. A computer readable storage medium may be, by way of non-limiting example, an electronic, magnetic, optical, electromagnetic, infrared, or semiconductor system, apparatus, or device, or any suitable combination of the foregoing. More specific non-limiting examples of the computer readable storage medium may include an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that may contain, or store a program for use by or in connection with an instruction execution system, apparatus, or device.

A computer readable signal medium may include a propagated data signal with computer readable program code embodied therein, by way of non-limiting example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that may communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, and the like, or any suitable combination thereof. Computer program code for carrying out operations for aspects of various embodiments may be written in any combination of one or more programming languages, including an object oriented programming language such as Java, Smalltalk, C++ or the like and conventional procedural programming languages, such as the C programming language or similar programming languages. The program code may also be written in a specialized language. The program code may execute entirely on the user's computer, partly on the user's computer, as a stand-alone software package, partly on the user's computer and partly on a remote computer, or entirely on the remote computer or server. The remote computer may be connected to the user's computer through any type of network, including a local area network (LAN) or a wide area network (WAN), or the connection may be made to an external computer (by way of non-limiting example, through the Internet using an Internet Service Provider).

As used herein, “tissue” refers to any natural or artificial tissues, including but not limited to skin, bone, muscle or other bodily tissues as would be understood to be bodily tissues by one of ordinary skill in the art, that is part of the body of a patient or body, body part of a patient or body, or organ for transplant.

### EMBODIMENTS OF THE APPLICATION

The application provides a surgical planning and navigation system comprising:

a) a computer capable of displaying various informational images related to any specific voxel in a body part of a patient for clinical diagnosis and surgical navigation;

b) optional stationary MRI compatible fiducial markers attached to rigid and unaffected tissue outside the area of surgical intervention of said patient, wherein fiducial markers enable the computer to co-register the various informational images related to any specific voxel in said body part from the images acquired prior, during, and after the surgical intervention promptly without considering defects produced by the surgical intervention;

c) a probe that is connected to the computer such that the computer can navigate the probe to a specific voxel; and

d) one or more electrodes, catheters, optical fibers or other investigational or treatment devices directed via the probe to a targeted voxel(s) in the tissue, wherein the investigational or treatment devices are capable of relaying real time functional information related to the targeted voxel(s) in the present surgical planning and navigation system.

The application provides the above surgical planning and navigation system, wherein the targeted voxel(s) in the tissue are within a region to be subjected to surgical intervention.

The application provides the above surgical planning and navigation system, wherein the surgical intervention is planned for the treatment of epilepsy, tumor, stroke or other diseases which requires the guidance from medical images.

The application provides any of the above surgical planning and navigation systems, wherein the fiducial markers are made of a non-paramagnetic material.

The application provides any of the above surgical planning and navigation systems, wherein the non-paramagnetic material is gold.

The application provides any of the above surgical planning and navigation systems, wherein the real time information related to the targeted voxel(s) is related to blood flow, oxygenation, metabolism, or other data related to physiological function of the tissue.

The application provides any of the above surgical planning and navigation systems, wherein the real time information related to the targeted voxel(s) in the tissue provides guidance to an operator of the system for planning surgical intervention of the tissue.

The application provides any of the above surgical planning and navigation systems, wherein during the surgical intervention of the tissue, the tissue is scanned using MRI.

The application provides any of the above surgical planning and navigation systems, wherein the MRI scans of the tissue can be pre-operative, intra-operative or post-operative scans are used to pre-surgical planning and to monitor the progress the planned surgical intervention of the tissue.

The application provides any of the above surgical planning and navigation systems, wherein the monitoring of the progress of the planned surgical intervention of the tissue guides the operator of the system during the planned surgical intervention of the tissue.

The application provides any of the above surgical planning and navigation systems, wherein the fiducial markers remain attached to the rigid and unaffected tissue outside the area of surgical intervention of the patient throughout the planned surgical intervention of the tissue for use in pre-operative, intra-operative or post-operative MRI scan.

The application provides any of the above surgical planning and navigation systems, wherein the post-operative MRI scans of the tissue are used to confirm the success of the planned surgical intervention of the tissue.

The application provides any of the above surgical planning and navigation systems, wherein the fiducial markers and further scans using MRI are used in the planning of additional planned surgical intervention of the tissue.

The application provides any of the above surgical planning and navigation systems, wherein fiducial makers are utilized for prompt co-registration alignment between pre-intervention images and post-intervention images without considering the image changes resultant of the surgical intervention.

The application provides any of the above surgical planning and navigation systems, wherein the patient is a pediatric patient.

The application provides a method of treating epilepsy, tumor, stroke or other diseases which requires the guidance from medical images in a patient using any of the above surgical planning and navigation systems.

The application provides the above method of ameliorating epilepsy, tumor, stroke or other diseases which requires the guidance from medical images in a patient using any of the above surgical planning and navigation systems.

The application provides any of the above methods of preventing future epileptic episodes in a patient using any of the above surgical planning and navigation systems.

The application provides any of the above methods of using any of the above surgical planning and navigation systems for the treatment, amelioration, or prevention of epileptic episodes in a patient.

The application provides any of the above methods, wherein the patient is a pediatric patient of age 5 or younger.

The application provides a computer-implemented surgical navigation planning method comprising:

a) providing a plurality of informational images associated with the same body part of a patient.

The application provides the above computer-implemented surgical navigation planning method, further comprising:

b) pre-processing various input imaging sources by converting them into each 3D image volumes with assigned attribute settings in a uniform coordinate system.

The application provides the above computer-implemented surgical navigation planning method, further comprising:

c) aligning all input image volumes to a base image volume through spatial co-registration procedure.

The application provides the above computer-implemented surgical navigation planning method, further comprising:

d) processing an image volume if needed through segmentation or creating activation map for highlighting significant regions thereof developing various aspects of each image volume.

The application provides the above computer-implemented surgical navigation planning method, further comprising:

e) treating each 3D image volume as an individual module with attribute settings. The integrated display is implemented by superimposing them according to their attribute settings and rending them in 2D or 3D views.

The application provides the above computer-implemented surgical navigation planning method, further comprising:

f) allowing the integrated display manipulation by adjusting the attribute settings.

The application provides the above computer-implemented surgical navigation planning method, further comprising:

g) planning a surgery based on the integrated display by creating new image volumes or modifying existing image volumes for displaying a trajectory of a planned resection margin or showing physiological/pathological properties of a location.

The application provides the above computer-implemented surgical navigation planning method, further comprising:

h) outputting integrated information including surgical plan into a surgical planning and navigation system or files.

The application provides the above computer-implemented surgical navigation planning method, further comprising:

i) implifying display setting for surgical planning and navigation system by using only one of attributes for promptly switching desired display during operation.

The application provides a method for planning the placement of an investigational or treatment device via the probe to a specific targeted voxel(s) in the tissue, comprising:

a) analyzing information of at least part of the tissue of the patient to determine if at least one specific or critical region or structure lies within a region of interest, said region of interest within a predetermined distance of a planned trajectory of the device in the tissue.

The application provides the above method, further comprising:

b) using a processor to assess a level of risk to the at least one specific or critical region or structure that is within the region of interest, wherein levels of risk are determined based on risk due to at least one of patient-specific physiological characteristics of the specific region or structure of the tissue, and wherein the levels of risk are determined based on at least one of:
i) risk due to anatomical, functional and/or physiological characteristics of the specific region or structure of the tissue, including risk of harming the tissue including the risk of crossing the trajectory of the device in the brain with a critical region for brain functioning, and/or risk of unsuccessful treatment including the risk of removal of critical tissue;
ii) risk due to a predefined treatment plan, said risk due to the predefined treatment plan including a proximity of a treatment approach or trajectory to a risk structure, the proximity of two or more different treatment approaches or trajectories to each other, and/or weighting the risk of the predefined treatment approach; or
iii) risk due to a predefined setup or configuration of a surgical planning and navigation system; and
iv) displaying borders along or around the specific or critical region or structure, wherein when planning the trajectory to cross at least one border, a warning signal is output or the trajectory is prevented from crossing the border.

The application provides the above method, wherein analyzing information includes automatically detecting the information of the at least part of the internal structure of the tissue or brain tissue, wherein said information includes at least one of anatomical, functional, angiographical and/or physiological structures of the tissue or brain tissue.

The application provides the above method, wherein automatically detecting includes automatically detecting structures that are manually outlined.

The application provides the above method, wherein the anatomical structures are obtained using at least one of Magnetic Resonance Imaging (MRI), X-Ray Imaging, Computer Tomography (CT) Imaging, or Ultra Sound Imaging, the physiological structures are determined using at least one of MR-DTI, MR-DCE, perfusion imaging from MR or CT, PET, SPECT, or MEG, and the functional structures are determined using at least one of fMRI, PET, brain-mapping, or EEG.

The application provides the above method, further comprising automatically displaying, classifying and/or assessing in terms of levels of risk, spatial location, potential adverse effect and/or proximity to the trajectory, the specific or critical region or structure in the region of interest.

The application provides the above method, further comprising defining the region of interest around the trajectory based on a user entry, or automatically or semi-automatically defining the region of interest based on patient information.

The application provides the above method, further comprising identifying in the information of at least a part of the internal structure of the tissue or brain tissue the specific or critical region or structure, and segmenting the specific or critical region or structure from the information of the internal structure of the tissue or brain tissue.

The application provides the above method, further comprising registering the segmented specific or critical region or structure with an image of the part of the internal structure of the tissue or brain tissue and overlaying the segmented specific or critical region or structure with the image.

The application provides the above method, further comprising using at least one of MRI diffusion scans, MRI-contrast enhanced scans, DTI, MRI dynamic contrast enhanced scans, CT perfusion scans, MRI T1w scans, MRI T2w scans, MRI proton density scans, MRI spectroscopy scans, PET scans, SPECT scans, molecular imaging, CT, X-ray, ultrasound, elastography, time series imaging, biopsies and/or tissue analysis to obtain the specific or critical region or structure.

The application provides the above method, further comprising displaying the specific or critical region or structure together with the planned trajectory on the image of the internal structure of the tissue or brain tissue.

The application provides the above method, wherein weighting the risk of the predefined treatment approach includes weighting the risk of a depth electrode plan or trajectory that is at least a predetermined distance away from a region critical for proper tissue or brain tissue functioning.

The application provides the above method, further comprising separately displaying levels of risk and/or displaying the levels of risk in the specific or critical region or structure.

The application provides the above method, further comprising determining trajectories that do or do not cross regions of a predetermined level of risk.

The application provides the above method, further comprising providing haptic feedback to a guiding device, whereby the haptic feedback is based upon a proximity between a surgical tool moving in a known relationship to the tissue or brain tissue and regions of risk.

The application provides the above method, further comprising providing a warning regarding a potential adverse effect of the planned placement of the device.

The application provides the above method, further comprising providing quantitative information regarding the proximity of the specific or critical region or structure with respect to one or more trajectories.

The application provides the above method, wherein the warning is an audible warning or a visual warning.

The application provides the above method, further comprising using the planned trajectory for at least one of stereotactic biopsies, electrode placement, catheter placement, cannula placement, stimulator placement, shunt placement, or surgical implant placement.

The application provides the above method, further comprising delivering electric energy to a region or structure based on a proximity of the delivery device to the specific or critical region or structure.

The application provides a method for planning the placement of an investigational or treatment device such as electrodes/catheters in the tissue or brain tissue of a pediatric patient, said electrode/catheter having a specific rigidity chosen from various types of electrodes stored in a database, comprising:

i) analyzing information of at least part of the internal structure of the tissue or brain tissue of the patient to determine if at least one specific or critical region or structure lies within a region of interest, said region of interest within a predetermined distance of a planned trajectory of the electrode in the tissue/brain;

ii) using a processor to assess a level of risk to the at least one specific or critical region or structure that is within the region of interest, wherein the level of risk and the rigidity of the electrode/catheter are determined based on risk due to patient-specific tissue density of a specific region or structure of the tissue or brain tissue and wherein the levels of risk are determined based on at least one of:

risk due to anatomical, functional and/or physiological characteristics of the specific region or structure of the brain, including risk of harming the tissue or brain tissue, and/or risk of unsuccessful treatment including the risk of crossing the trajectory of the device in the tissue or brain tissue or risk due to a predefined treatment plan, said risk due to the predefined treatment plan including a proximity of a treatment approach or trajectory to a risk structure, the proximity of two or more different treatment approaches or trajectories to each other, and/or weighting the risk of the predefined treatment approach; or

iii) risk due to a predefined setup or configuration of a surgical planning and navigation system; and

iv) displaying borders along or around the specific or critical region or structure, wherein when planning the trajectory to cross at least one border, a warning signal is output or the trajectory is prevented from crossing the border.

The application provides a surgical planning and navigation system comprising:

a) a computer capable of displaying various informational images related to any specific voxel in a the brain of a patient for clinical diagnosis and surgical navigation;

b) optional stationary MRI compatible fiducial markers attached to rigid and unaffected tissue outside the area of surgical intervention of said patient, wherein the fiducial markers enable the computer to co-register the various informational images related to any specific voxel in said brain from the images acquired prior, during, and after surgical intervention promptly without considering defects produced by the surgical intervention;

c) a probe that is connected to the computer such that the computer can navigate the probe to a specific voxel; and

d) one or more electrodes, catheters, optical fibers or other investigational or treatment devices directed via the probe to a targeted voxel(s) in the brain, wherein said investigational or treatment devices are capable of relaying real time functional information related to the targeted voxel(s) in the present surgical planning and navigation system.

The application provides the above surgical planning and navigation system, wherein the targeted voxel(s) in the brain are within a region to be subjected to surgical intervention.

Surgical intervention comprises resection, biopsies, implantation and treatment delivery, etc.

The application provides either of the above surgical planning and navigation systems, wherein the surgical intervention is planned for the treatment of epilepsy, tumor, stroke or other diseases which requires the guidance from medical images.

The application provides any one of the above surgical planning and navigation systems, wherein the fiducial markers are made of a non-paramagnetic material.

The application provides any one of the above surgical planning and navigation systems, wherein the non-paramagnetic material is gold.

The application provides any one of the above surgical planning and navigation systems, wherein the real time information related to the targeted voxel(s) is related to blood flow, oxygenation, metabolism, or other data related to physiological functions of the brain.

The application provides any one of the above surgical planning and navigation systems, wherein the real time functional information related to the targeted voxel(s) in the brain provides guidance to an operator of the system for planning surgical intervention of the brain tissue.

The application provides any one of the above surgical planning and navigation systems, wherein during the surgical intervention, the brain tissue is scanned using intra-operative MRI.

The application provides any one of the above surgical planning and navigation systems, wherein the intra-operative MRI scans of the brain tissue are used to monitor the progress the planned surgical intervention of the brain tissue.

The application provides any one of the above surgical planning and navigation systems, wherein the monitoring of the progress the planned surgical intervention of the brain tissue guides the operator of the system during the planned surgical intervention of the brain tissue.

The application provides any one of the above surgical planning and navigation systems, wherein the fiducial markers remain attached to the rigid and unaffected tissue outside surgical intervention of the patient throughout the planned surgical intervention of the brain tissue for use in pre-operative, intra-operative or post-operative MRI scan.

The application provides any one of the above surgical planning and navigation systems, wherein the post-operative MRI scans of the tissue are used to confirm the success of the planned surgical intervention of the brain tissue.

The application provides any one of the above surgical planning and navigation systems, wherein the fiducial markers and further scans using MRI are used in the planning of additional planned surgical intervention of the brain tissue.

The application provides any one of the above surgical planning and navigation systems, wherein fiducial makers are utilized for prompt co-registration alignment between pre-intervention images and post-intervention images without considering the image changes resultant of the surgical intervention.

The application provides a method of treating epilepsy, tumor, stroke or other diseases which requires the guidance from medical images in a patient using the surgical planning and navigation system of any one of the above surgical planning and navigation systems.

The application provides a method of ameliorating epilepsy, tumor, stroke or other diseases which requires the guidance from medical images in a patient using the surgical planning and navigation system of any one of the above surgical planning and navigation systems.

The application provides a method of preventing future epileptic episodes in a patient using the surgical planning and navigation system of any one of the above surgical planning and navigation systems.

The application provides the use of the surgical planning and navigation system of any one of the above surgical planning and navigation systems for the treatment, amelioration, or prevention of epileptic episodes.

The application provides any system, method, or use as described herein.

The application provides a computer-implemented surgical planning and navigation planning method comprising:

a) providing a plurality of informational images associated with the brain of a patient.

The application provides the above computer-implemented surgical planning and navigation planning method, further comprising:

b) pre-processing various input imaging sources by converting them into each 3D image volumes with assigned attribute settings in a uniform coordinate system.

The application provides the above computer-implemented surgical planning and navigation planning method, further comprising:

c) aligning all input image volumes to a base image volume through spatial co-registration procedures.

The application provides the above computer-implemented surgical planning and navigation planning method, further comprising:

d) processing an image volume as needed through segmentation or creating an activation map for highlighting significant regions therein and developing various aspects of each image volume.

The application provides the above computer-implemented surgical planning and navigation planning method, further comprising:

e) treating each 3D image volume as an individual module with attribute settings. The integrated display is implemented by superimposing them according to their attribute settings and rending them in 2D or 3D views.

The application provides the above computer-implemented surgical planning and navigation planning method, further comprising:

f) allowing the integrated display manipulation by adjusting the attribute settings.

The application provides the above computer-implemented surgical planning and navigation planning method, further comprising:

g) planning a surgery based on the integrated display by creating new image volumes or modifying existing image volumes for displaying a trajectory of a planned resection margin or showing physiological/pathological properties of a location.

The application provides the above computer-implemented surgical planning and navigation planning method, further comprising:

h) outputting integrated information including surgical planning into a surgical planning and navigation system or file.

The application provides the above computer-implemented surgical planning and navigation planning method, further comprising:

i) simplifying display settings for the surgical planning and navigation system by using only one of the attributes for promptly switching the desired display during operation

The application provides a method for planning the placement of an investigational or treatment device via a probe to a specific targeted voxel(s) in the tissue or brain tissue of a patient, comprising:

a) analyzing information of at least part of the brain of the patient to determine if at least one specific or critical region or structure lies within a region of interest, said region of interest within a predetermined distance of a planned trajectory of the device in the brain.

The application provides the above method, further comprising:

b) using a processor to assess a level of risk to the at least one specific or critical region or structure that is within the region of interest, wherein levels of risk are determined based on risk due to at least one of patient-specific physiological characteristics of the specific region or structure of the brain, and wherein the levels of risk are determined based on at least one of:
i) risk due to anatomical, functional and/or physiological characteristics of the specific region or structure of the brain, including risk of harming the brain including the risk of crossing the trajectory of the device in the brain with a critical region for brain functioning, and/or risk of unsuccessful treatment including the risk of removal of critical brain tissue;
ii) risk due to a predefined treatment plan, said risk due to the predefined treatment plan including a proximity of a treatment approach or trajectory to a risk structure, the proximity of two or more different treatment approaches or trajectories to each other, and/or weighting the risk of the predefined treatment approach; or
iii) risk due to a predefined setup or configuration of a surgical planning and navigation system; and
iv) displaying borders along or around the specific or critical region or structure, wherein when planning the trajectory to cross at least one border, a warning signal is output or the trajectory is prevented from crossing the border.

The application provides either of the above methods, wherein analyzing information includes automatically detecting the information of the at least part of the internal structure of the tissue brain or brain tissue, wherein said information includes at least one of anatomical, functional, angiographical and/or physiological structures of the tissue or brain tissue.

The application provides any of the above methods, wherein automatically detecting includes automatically detecting structures that are manually outlined.

The application provides any of the above methods, wherein the anatomical structures are obtained using at least one of Magnetic Resonance Imaging (MRI), X-Ray Imaging, Computer Tomography (CT) Imaging, or Ultra Sound Imaging, the physiological structures are determined using at least one of MR-DTI, MR-DCE, perfusion imaging from MR or CT, PET, SPECT, or MEG, and the functional structures are determined using at least one of fMRI, PET, brain-mapping, or EEG.

The application provides any of the above methods, further comprising automatically displaying, classifying and/or assessing in terms of levels of risk, spatial location, potential adverse effect and/or proximity to the trajectory, the specific or critical region or structure in the region of interest.

The application provides any of the above methods, further comprising defining the region of interest around the trajectory based on a user entry, or automatically or semi-automatically defining the region of interest based on patient information.

The application provides any of the above methods, further comprising identifying in the information of at least a part of the internal structure of the tissue or brain tissue the specific or critical region or structure, and segmenting the specific or critical region or structure from the information of the internal structure of the tissue or brain tissue.

The application provides any of the above methods, further comprising registering the segmented specific or critical region or structure with an image of the part of the internal structure of the brain and overlaying the segmented specific or critical region or structure with the image.

The application provides any of the above methods, further comprising using at least one of MRI diffusion scans, MRI-contrast enhanced scans, DTI, MRI dynamic contrast enhanced scans, CT perfusion scans, MRI T1w scans, MRI T2w scans, MRI proton density scans, MRI spectroscopy scans, PET scans, SPECT scans, molecular imaging, CT, X-ray, ultrasound, elastography, time series imaging, biopsies and/or tissue analysis to obtain the specific or critical region or structure.

The application provides any of the above methods, further comprising displaying the specific or critical region or structure together with the planned trajectory on the image of the internal structure of the tissue or brain tissue.

The application provides any of the above methods, wherein weighting the risk of the predefined treatment approach includes weighting the risk of a surgical intervention/implantation plan or trajectory that is at least a predetermined distance away from a region critical for proper tissue or brain tissue functioning.

The application provides any of the above methods, further comprising separately displaying levels of risk and/or displaying the levels of risk in the specific or critical region or structure.

The application provides any of the above methods, further comprising determining trajectories that do or do not cross regions of a predetermined level of risk.

The application provides any of the above methods, further comprising providing haptic feedback to a guiding device, whereby the haptic feedback is based upon a proximity between a surgical tool moving in a known relationship to the brain and regions of risk.

The application provides any of the above methods, further comprising providing quantitative information regarding the proximity of the specific or critical region or structure with respect to one or more trajectories.

The application provides any of the above methods, further comprising providing a warning regarding a potential adverse effect of the planned placement of the device.

The application provides the above method, wherein the warning is an audible warning or a visual warning.

The application provides any of the above methods, further comprising using the planned trajectory for at least one of stereotactic biopsies, electrode placement, catheter placement, cannula placement, stimulator placement, shunt placement, or surgical implant placement.

The application provides any of the above methods, further comprising delivering any type of energy to a region or structure based on a proximity of the delivery device to the specific or critical region or structure.

The application provides a method for planning the placement of an investigational or treatment device such as an electrode/catheter in the tissue or brain tissue of a patient, said electrode/catheter having a specific rigidity chosen from various types of electrode/catheter stored in a database, comprising:

i) analyzing information of at least part of the internal structure of the tissue or brain tissue of the patient to determine if at least one specific or critical region or structure lies within a region of interest, said region of interest within a predetermined distance of a planned trajectory of the electrode/catheter in the tissue or brain tissue;

ii) using a processor to assess a level of risk to the at least one specific or critical region or structure that is within the region of interest, wherein the level of risk and the rigidity of the electrode/catheter are determined based on risk due to patient-specific tissue density of a specific region or structure of the tissue or brain tissue and wherein the levels of risk are determined based on at least one of:

risk due to anatomical, functional and/or physiological characteristics of the specific region or structure of the brain, including risk of harming the tissue or brain tissue, and/or risk of unsuccessful treatment including the risk of crossing the trajectory of the device in the tissue or brain tissue or risk due to a predefined treatment plan, said risk due to the predefined treatment plan including a proximity of a treatment approach or trajectory to a risk structure, the proximity of two or more different treatment approaches or trajectories to each other, and/or weighting the risk of the predefined treatment approach; or

iii) risk due to a predefined setup or configuration of a surgical planning and navigation system; and

iv) displaying borders along or around the specific or critical region or structure, wherein when planning the trajectory to cross at least one border, a warning signal is output or the trajectory is prevented from crossing the border.

According to one aspect of the invention, there is provided a method for planning the placement of a device into the tissue or brain tissue or the movement of the device in the tissue or brain tissue. At least a part of the internal structure or the whole internal structure of the tissue or brain tissue is analyzed to determine if a critical region of the tissue or brain tissue, such as a critical anatomical structure (e.g., ventricles) or physiological or functional structure, which preferably should not be harmed, is in or near the planned trajectory of the device or within a region of interest around the planned trajectory. The device can be any device used for medical examination or treatment and, for example, can be an electrode, biopsy needle or an intra-cranial catheter. These devices should be safely introduced and placed into the tissue or brain tissue and moved therein without harming the patient. For example, the devices should not cross an optic nerve or other important anatomical or functional risk structure, while simultaneously considering the success of a medical examination or therapy by reaching a specific tissue or brain tissue structure.

In other words, the devices such as depth electrodes preferably are located such that a target area is optimally reached at the intended location and trajectory of the electrode or catheter. The levels of risk resulting from a specifically planned trajectory or the difficulty in carrying out a surgical plan or medical examination associated with planned stereotactic trajectories can be assessed and related to a specific plan or trajectory of a device to be introduced into, placed in and/or moved in the brain of a patient. More particularly, when considering a plan for placing a device in a brain, the proximity of a treatment approach, e.g., a trajectory of the device, to risk structures can be more accurately determined and the risk level of a particular treatment approach can be accordingly weighted. In this manner, for example, a plan can be drawn up such that a device such as an electrode or catheter to be more accurately placed in the tissue or brain tissue has a predetermined minimum distance from certain critical structures and, for example, stays at least a predetermined distance from critical structures or areas required for normal functionality.

The three dimensional structure of the tissue or brain tissue can be considered and inaccuracies of the planning and placement approach, which may result, for example, from restricted image resolution, non-perfect patient registration, instability or flexibility of a catheter and so on, can be determined or anticipated for consideration. Then, the risk of reaching or not reaching a specific structure can be rated. For example, it can be considered that an electrode or catheter having a high flexibility may be deflected to cross a critical region, which may increase the determined level of risk. Furthermore, it can be made more possible to stay away from a critical area, by a predetermined distance of, for example, 5 mm at the point of entry, whereas further down the trajectory, the predetermined distance of for example 5 mm may be insufficient if an unstable electrode or catheter is used (e.g., a catheter that is likely to bend toward the risk structure).

Furthermore, inaccuracy based on the navigational approach, can be considered for planning the trajectory to guarantee that a specific structure is or is not reached when introducing the device. Image resolution and data accuracy can be taken into consideration to determine the accuracy of the method and thus the risk of reaching risk structures.

The risk due to a particular setup or configuration of a surgical planning and navigation system can be considered. For delivering a probe, the holding device used in surgery bears inherent inaccuracies, e.g., if a surgeon uses a pointer to pass a catheter along a desired trajectory, the anticipated inaccuracies are higher than with a rigidly mounted holding device, which is, however, unable to be used in children under 5 years of age due to malleability of the young skull structure which render it unable to withstand the pressure of a rigid frame based approach. Additionally, when the holding device is fixed far away from the entry point, the risk of inaccurate placement is elevated versus fixing the holding device close to the entry point. These inaccuracies can be determined or estimated and can be used for planning the movement of a device into or in a brain. An electrode or catheter having high flexibility may be deflected into or towards a structure of risk, which would increase the respective level of risk, whereas a stiff or hard electrode or catheter may decrease this level of risk. However, for example, in vascular structures, the stiff catheter may increase the risk of harming vascular structures compared to a more flexible catheter. Also, an experienced or a good surgeon may decrease the calculated level of risk, whereas an inexperienced surgeon may increase it.

The structures of the tissue or a part of the brain, such as the brain, can be detected automatically to detect the anatomical, functional and/or physiological structures by using, for example, known segmentation techniques. Furthermore, it is also possible to use manually outlined structures that can be identified by an experienced surgeon. These structures can be manually or automatically related to different levels of risk.

The analysis of the brain or a part thereof, especially structural data, can be performed on the basis of anatomical data obtained by MRI, CT, X-Ray, ultrasound, physiological data obtained by MR-DTI, MR-DCE, perfusion imaging from MR or CT, PET, SPECT, MEG, functional data obtained by fMRI, PET, brain-mapping, EEG and/or angiographic data which enables the visualization of veins and/or arteries.

The method described herein enables automatic detection of anatomical, functional, angiographic and/or physiological structures of the tissue, particularly the brain and/or manually outlined structures. The structures can be identified in the internal structure of the brain or in images and/or data of the internal structure of the brain, and can be automatically segmented from said images. These structures can be either manually or automatically related to different levels of risk.

For example, after entering a trajectory for biopsy, catheter placement, shunt placement or stimulator placement based on the patient-specific input data and/or automatically and/or manually outlined structures, the method preferably can check for existence of critical structures in a selected area around the trajectory. The volume or region of interest can be defined as a default value or by a predetermined value around or close to the trajectory or can be defined manually by the user, or can be automatically or semi-automatically defined using information about patients and/or treatments and or the treating physicians experience. Critical regions or structures appearing within this volume can be automatically displayed and/or classified in terms of risk levels. Different risk levels can be defined in terms of proximity or likelihood of reaching this structures or can be determined based on proximity to the trajectory, anatomical characteristics, functional characteristics as well as physiological characteristics of the critical structure. Also close proximity between different trajectories can be included as a risk. In general, these characteristics can be used to assess potential risk levels for affecting functionality or any adverse effect due to the crossing of the trajectory in the respective region. Information of the identified critical structure can be displayed in terms of: spatial location, level of risk/potential adverse effect, and proximity to the trajectory.

Regarding spatial location, the critical area can be obtained by patient-specific image data and/or can be outlined manually by the user. In one embodiment, the segmented critical structure can be registered with an image of the part of the brain and might then be overlaid on the co-registered subject image. Levels of risk can be displayed, for example, on a separate window and/or by color classification of the segmented critical structure. Warnings about the potential adverse effect from crossing this structure also can be displayed. Quantitative information regarding the proximity of the critical structure with respect to one or more trajectories can also be provided.

An application is for the use of described information for navigation systems. Virtual boarders can be created along or around critical regions that do not allow the user or warn the user if areas behind those walls or boarders are entered.

Suggestions for trajectories that do (or do not) cross regions of predefined or predetermined risk levels can be created or determined. For example, the trajectory can be planned such that it only crosses areas or locations of a predetermined risk level but does not cross areas with a higher risk level. Furthermore, the distance from said areas which are not to be crossed by the trajectory can be related to or proportional to the height or extent of said risk level.

Information can be used for creation of suggestions for trajectories that are placed in a predefined distance to areas of predefined risk levels. This distance can be the same constant distance for every critical structure with a risk level above a predetermined risk level or can be related to or proportional to the extent of each level of risk.

The provision of haptic feedback in a delivery device, whereby the feedback may be based upon the proximity between a surgical tool moving in a known relationship to the regions of risk. For example, the distance of a surgical tool to critical structures can be determined repeatedly. Based on said distance and the risk level of each critical region, a strong or weak haptic feedback can be provided to make the navigation or assisting system change the direction or position of the surgical tool.

The directed delivery of electrical energy, e.g., by neurostimulation, may be based on the knowledge of the proximity of a delivery device to risk structures. Further, the directed delivery of a therapeutic agent may be based on the knowledge of the flow patterns into nearby structures of risk.

The method described herein enables the physician to focus on finding the optimal trajectory for a treatment or medical examination by providing information in terms of anatomical, functional and/or physiological information and/or based on warnings about the potential adverse effects when chosen trajectories cross a critical area or region as outlined. Transferring the information to a surgical planning and navigation system increases safety for the patient by excluding areas associated with high-risk levels.

In accordance with another aspect of the invention, there is provided a method for planning an infusion, e.g., for administering a substance or an active agent, in particular for injection into tissue, preferably into a predetermined tissue structure or tissue volume, wherein patient data or parameters obtained from the patient are captured. Known magnetic resonance imaging methods (MRI), computer tomography (CT) methods, x-ray methods, ultrasound methods or other suitable methods, which enable the spatial structure of a brain, in particular of a tissue structure, to be detected and displayed and/or functional data, such as for example patient-specific diffusion and perfusion properties, to be obtained, can be used in this respect. Infusion can be planned as described above using the patient data (e.g., the catheter to be used is suitably selected, the catheter is positioned with respect to the insertion location and depth of penetration, the infusing medium is selected and if necessary modified, for example thinned, and the pressure gradient over time with which the infusing medium is to be delivered through the catheter is predetermined by taking into account various selectable pre-set figures, such as for example patient data or also parameters of available substances to be administered, parameters of the catheters which may be used and possibly of a pump which may be used. The aim of this selecting and setting is to inject a defined quantity of a substance to be administered into a target tissue volume, in order to obtain a particular concentration there, wherein as little of the substance to be administered as possible is to be introduced into non-target tissue.

The captured patient data may be used to position the device(s), for example one or more depth electrodes or catheters, wherein it is determined from these patient data where exactly in the patient's brain a tissue volume to be treated, for example epilepsy or brain tumor, is situated. Using this information, a suitable electrode or catheter can be selected, for example from an available data base, by an operator or automatically, and possibly modified by post-processing, for example trimming the length of the electrode or catheter, in accordance with application or patient specifications, for example with respect to the desired depth of penetration into the tissue or the exact planned position of the depth electrode or catheter. Furthermore, a suitable point for inserting a catheter or how the catheter should be placed in the tissue can be determined such that the infusion debilitates as little healthy tissue and as much non-healthy tissue as possible.

Known positioning methods can advantageously be used. For example, reflecting markers that are attached to the catheter and detected by IR cameras can be used in order to locate the catheter at a desired position on the patient. To this end, fiducial markers also may be attached to the patient, wherein the markers serve as a reference and through which a patient coordinate system can be determined that enables an electrode or catheter to be placed at a particular determined point.

Patient-specific parameters preferably are determined using the captured patient data for planning the infusion, for example the tissue or brain structure in the area of the tissue to be treated by the infusion. It is particularly advantageous to determine the tissue density, the distribution of particular tissue structures, or the blood flow through a particular area of tissue, as patient parameters. Patient parameters may be obtained both directly from the captured patient data as well as from databases or from a combination of values stored in databases together with the captured patient data and furthermore in combination with real time patient data during operation. Thus, values which may be used as patient parameters for planning implantation of a depth electrode, or for an infusion, can be stored, for example values relating to usual blood flow through particular areas of tissue, the diffusion and perfusion behavior of selected substances in the tissue under consideration, and values relating to tissue behavior after a known substance has been delivered, for example swelling of the tissue or metabolic reactions.

Furthermore, retro or inverse planning also can be performed, wherein for example treatment data defined by an operator may be pre-set, such as for example the target volume to be treated, advantageously together with high-risk structures such as for example nerve tracts that should not be compromised by an electrode or catheter, levels of risk of regions of the tissue, and details of the type of tissue to be treated. In this way, either automatically or by interaction with the operator, for example by displaying a selection menu, the course of the electrode or catheter can be established (e.g., one or more types of electrode or catheter can be selected together with suitable media, the arrangement(s) of electrode or catheter can be determined with respect to position and/or depth of penetration and the parameters of the electrode or catheter can be set) in order to enable an optimal resection surgery for the given target volume.

The planning methods described above, in particular the selecting of individual parameters and the segmentation of critical regions of the tissue and the determination of levels of risk in said region, can be performed: fully automatically using, for example, values stored in data bases; semi-automatically, for example by selections made by an operator from a displayed menu; or manually, for example through parameter values input by an operator. In this respect, suitable computers can be advantageously used, together with input and output elements, for example display elements displaying elements to be selected, tissue structures, calculated concentration distributions of the infusing medium and other information.

In accordance with another aspect of the invention, there is provided a computer program which, when loaded or running on a computer, performs the method described above or parts of it. Equally, the present invention relates to a storage medium for such a program or to a computer program product comprising the aforementioned program.

A device for planning a resection surgery, comprises a planning system including a computer system, preferably having input and output devices and corresponding software. In this respect, a monitor can be advantageously provided for displaying elements pre-set by the computer from databases or values determined from calculations or spatial distributions.

A navigation system can be provided, including, for example, reflecting markers, LEDs or coils attached to elements to be positioned and IR cameras or magnetic field generators, with which a catheter on a brain, for example, can be positioned using a suitable, known software and hardware.

Generally, the device can include elements, devices and systems with which the steps of the method described above may be performed.

In accordance with another aspect of the invention, there is provided a resection surgery method, wherein resection surgery is preferably prepared as described above and the resection medium is then introduced into the tissue or brain tissue.

Verification can be performed continuously or at particular intervals in time during the resection surgery. The targeting of the resection surgery in the tissue during or after the surgical process can be determined using a suitable data capture or representation system. Magnetic resonance imaging, x-ray based methods, or ultrasound methods, for example, may be used in this respect, wherein it may be advantageous to add a contrast medium in order to clearly establish or measure the placement of electrodes, catheters or surgical instrumentation in the brain tissue.

Advantageously, any surgical deviation can be determined, verified and the correction made in real time, such that the surgery can be controlled via a back-coupling (e.g., feedback) to obtain the desired successful resection.

In accordance with a further aspect of the invention, there is provided a computer program which, when loaded or when running on a computer, performs the method described above. Equally, the present invention relates to a storage medium for such a program or to a computer program product comprising the aforementioned program.

According to a further aspect of the invention, there is provided a device for carrying out an infusion method as described above, comprising a verification device for determining the spatial distribution of an infusing medium in a brain, in particular in an area of tissue. The verification device, for example, can be a magnetic resonance or nuclear spin resonance, x-ray, or ultrasound system with which the infusing medium or its distribution and concentration in the tissue can be detected.

A computer system can be provided with a display device to enable evaluation of the determined spatial distribution of instrumentation in the tissue, to establish a deviation from a previously established surgical plan and possibly to automatically alter the infusion parameters or propose such a change to an operator, in order to modify the surgery such that it can be carried out as planned.

When a deviation from a given surgical plan is established during verification, the manner and magnitude of the change to the parameters can be advantageously determined using known action and function mechanisms. For example, the rate of delivery or the injection pressure can be reduced when it is established that the surgical plan is other than predicted.

The provision of haptic feedback in a delivery device, e.g., probe, robotic or assisting arm system, can be used, whereby the feedback may be based upon the proximity between a surgical tool moving in a known relationship to the navigation system and regions of risk. For example, the distance of a depth electrode to critical structures can be determined repeatedly. Based on said distance and the risk level of each critical region, a strong or weak haptic feedback can be provided to make the navigation system change the direction or position of the surgical tool.

The directed delivery of electrical energy, e.g., by neurostimulation, may be based on the knowledge of the proximity of a delivery device to risk structures. Further, the directed delivery of a therapeutic agent may be based on the knowledge of the flow patterns into nearby structures of risk.

The method described herein enables the physician to focus on finding the optimal trajectory for a treatment or medical examination by providing information in terms of anatomical, functional and/or physiological information and/or based on warnings about the potential adverse effects when chosen trajectories cross a critical area or region as outlined. Transferring the information to a surgical planning and navigation system increases safety for the patient by excluding areas associated with high-risk levels.

In accordance with another aspect of the invention, there is provided a method for planning a resection surgery, preferably into a predetermined tissue structure or tissue volume, wherein patient data or parameters obtained from the patient are captured. Known magnetic resonance imaging methods (MRI), computer tomography (CT) methods, x-ray methods, ultrasound methods or other suitable methods, which enable the spatial structure of a brain, in particular of a tissue structure, to be detected and displayed and/or functional data, such as for example patient-specific diffusion and perfusion properties, to be obtained, can be used in this respect. Infusion can be planned as described above using the patient data (e.g., the catheter to be used is suitably selected, the catheter is positioned with respect to the insertion location and depth of penetration, the infusing medium is selected and if necessary modified, for example thinned, and the pressure gradient over time with which the infusing medium is to be delivered through the catheter is predetermined by taking into account various selectable pre-set figures, such as for example patient data or also parameters of available substances to be administered, parameters of the catheters which may be used and possibly of a pump which may be used. The aim of this selecting and setting is to inject a defined quantity of the substance to be administered into a target tissue volume, in order to obtain a particular concentration there, wherein as little of the substance to be administered as possible is to be introduced into non-target tissue.

The captured patient data may be used to position the infusion device(s), for example one or more catheters, wherein it is determined from these patient data where exactly in the patient's brain a tissue volume to be treated, for example brain tumor, is situated. Using this information, a suitable catheter can be selected, for example from an available data base, by an operator or automatically, and possibly modified by post-processing, for example trimming the length of the catheter, in accordance with application or patient specifications, for example with respect to the desired depth of penetration into the tissue or the exact planned position of the catheter. Furthermore, a suitable point for inserting the catheter or how the catheter should be placed in the tissue can be determined such that the infusion debilitates as little healthy tissue and as much non-healthy tissue as possible.

Known positioning methods can advantageously be used. For example, reflecting markers that are attached to the catheter and detected by IR cameras can be used in order to locate the catheter at a desired position on the patient. To this end, markers also may be attached to the patient, wherein the markers serve as a reference and through which a patient coordinate system can be determined that enables the catheter to be placed at a particular determined point.

Patient-specific parameters preferably are determined using the captured patient data for planning the infusion, for example the tissue or brain structure in the area of the tissue to be treated by the infusion. It is particularly advantageous to determine the tissue density, the distribution of particular tissue structures, or the blood flow through a particular area of tissue, as patient parameters. Patient parameters may be obtained both directly from the captured patient data as well as from databases or from a combination of values stored in databases together with the captured patient data. Thus, values which may be used as patient parameters for planning a resection surgery can be stored, for example values relating to usual blood flow through particular areas of tissue, oxygen flow, metabolic reactions, for example, as values relating to tissue behavior after a depth electrode has been placed.

Catheter variables (i.e., variables specific to a catheter for insertion) may be used, wherein various types of catheters could be provided, for example in a database, and selections may be made from these catheter types. Catheter parameters relevant to the insertion, for example, can be the inner diameter of the catheter, surface finish, the material, in particular the rigidity of the catheter, the shape, the number and arrangement of outlets on the catheter or a known suitability of a particular type of catheter for a particular substance to be administered or a particular type of tissue or diseased tissue to be treated. In general, a number of catheters may also be used.

By using the patient parameters, parameters of the depth electrodes and/or catheter parameters cited above by way of example, individually or in combination, together with the captured patient data, a resection surgery to be performed can be planned, such that as large a proportion of diseases tissue as possible is removed, wherein as little of the properly functioning tissue as possible is salvaged. Thus, an electrode to be introduced into a tissue by a probe can be introduced into an area of tissue to be treated in the patient using a particularly suitable and correctly positioned type of navigational probe. Surrounding tissue is thus debilitated as little as possible.

Furthermore, retro or inverse planning can be performed, wherein for example treatment data defined by an operator may be pre-set, such as for example the target volume to be treated, advantageously together with high-risk structures such as for example nerve tracts that should not be compromised by the resection, levels of risk of regions of the tissue, and details of the type of tissue to be treated. In this way, either automatically or by interaction with the operator, for example by displaying a selection menu, the course of the resection can be established (e.g., one or more types of catheters or depth electrodes can be selected with respect to position and/or depth of penetration can be set) in order to enable an optimal resection surgery for the given target volume.

The planning methods described above, in particular the selecting of individual parameters and the segmentation of critical regions of the tissue and the determination of levels of risk in said region, can be performed: fully automatically using, for example, values stored in data bases; semi-automatically, for example by selections made by an operator from a displayed menu; or manually, for example through parameter values input by an operator. In this respect, suitable computers can be advantageously used, together with input and output elements, for example display elements displaying elements to be selected, tissue structures, and other information.

In accordance with another aspect of the invention, there is provided a computer program which, when loaded or running on a computer, performs the method described above or parts of it. Equally, the present invention relates to a storage medium for such a program or to a computer program product comprising the aforementioned program.

A device for planning a resection comprises a planning system including a computer system, preferably having input and output devices and corresponding software. In this respect, a monitor can be advantageously provided for displaying various elements pre-set by the computer from databases or values determined from predetermined or live calculations or spatial distributions.

A navigation system can be provided, including, for example, fiducial markers connected to the skull, with which an electrode, depth electrode, or catheter, for example, can be positioned using a suitable, known software and hardware.

Generally, the device can include elements, devices and systems with which the steps of the method described above may be performed.

In accordance with another aspect of the invention, there is provided a navigational method, wherein the resection is preferably prepared as described above after the depth electrode is successfully introduced into the brain or brain tissue.

Verification can be performed continuously or at particular intervals in time during the resection surgery. The depth electrode in the tissue during or after the resection process can be determined using a suitable data capture or representation system. Magnetic resonance imaging, x-ray based methods, or ultrasound methods, for example, may be used in this respect, wherein it may be advantageous to add a contrast medium in order to clearly establish or measure the effectiveness of the placement of the depth electrode in the brain tissue.

Preferably, deviations between the actual placement of the depth electrode in the tissue and the planning data (which may be determined before or during the placement) as determined from the verification process can be displayed. Advantageously, the placement parameters may be corrected, e.g., the navigation of delivery may be changed, to be able to correct for any deviation, determined during verification, from the planned navigation. If necessary, a catheter, electrode, or depth electrode can also be repositioned or exchanged.

Advantageously, the deviation can be determined, verified and the correction made in real time, such that the navigation and ultimate resection can be controlled via a back-coupling (e.g., feedback) to obtain the desired successful resection, i.e., to remove the given target area as desired.

In accordance with a further aspect of the invention, there is provided a computer program which, when loaded or when running on a computer, performs the method described above. Equally, the present invention relates to a storage medium for such a program or to a computer program product comprising the aforementioned program.

According to a further aspect of the invention, there is provided a device for carrying out a navigation method as described above, comprising a verification device for determining the spatial distribution of a depth electrode in a brain, in particular in an area of tissue. The verification device, for example, can be a magnetic resonance or nuclear spin resonance, x-ray, or ultrasound system with which the electrode in the tissue can be detected.

A computer system can be provided with a display device to enable evaluation of the determined navigational path of the depth electrode in the tissue, to establish a deviation from a previously established navigational plan and possibly to automatically alter the parameters or propose such a change to an operator, in order to modify the ultimate resection surgery such that it can be carried out as planned. To this end, systems can be provided that enable the accuracy of the navigational path to be optimized or changed and altered, if necessary, to obtain an accurate plan for the ultimate resection surgery as previously planned. When a deviation from a given navigational plan is established during verification, the manner and magnitude of the change to the resection parameters can be advantageously determined using known action and function mechanisms.

In one embodiment, the resection plan can be communicated via an interface to a navigation system, such as for example the VectorVision® system. The navigation system can be used to position the selected depth electrode or catheter at the given points in the brain based on the planning data. The electrodes or catheter(s) can be positioned automatically, for example using a robot, or manually with guidance from the navigation system (e.g., a display device showing whether the electrode or catheter is correctly positioned or still has to be moved in a particular direction). The results of the positioning and navigation may be output to a display device.

Once the electrodes or catheter(s) has/have been successfully positioned, data may be communicated to a navigation system (e.g., the resection parameters may be transferred to the navigation device or system and a command may be provided to instruct the surgeon where to conduct the resection). To this end, patient data are captured to determine the actual area within the brain that needs to be removed. Using the parameters set during resection planning, and the results of the simulation of the resection based on the plan, a comparison can be made between the actual necessary area to be removed and the predicted area to be removed. The comparison data can be communicated back, wherein the resection parameters can be altered as appropriate, preferably taking into account known action mechanisms in order to obtain the desired, planned resection result. The measured, actual area to undergo resection, preferably together with possible deviations and correcting methods, can again be output via a display to enable an operator, for example, to manually intercede in the resection method.

In one embodiment, patient data are captured using an imaging diagnostic method such as, for example, a magnetic resonance or nuclear spin resonance method, to obtain the current patient parameters (e.g., tissue density, blood flow, and the location of a tissue to be treated). Using the patient parameters determined in this way (e.g., data of levels of risk of critical regions) and with catheter parameters and/or parameters of the information obtained from electrodes, the resection can be planned and/or simulated. Based on the parameter data determined in this way, the resection plan is forwarded to a navigation system, which can be used to position the electrode or catheters on the patient as provided for in the resection plan.

In one embodiment, a system that may be used when planning and carrying out a resection in accordance with the invention. Patient data are obtained in a magnetic resonance or nuclear spin tomograph and forwarded to a planning system and to a navigation system. The electrode or catheters may be positioned at a desired point in the brain by the navigation system using, for example, known reflectors or markers attached to one or more catheters, positional data of the markers being captured by IR cameras. In order to carry out the resection, the planning system determines the suitable electrode or catheter parameters and parameters for the resection to be carried out, using patient parameters determined by predetermined, live, or post-operative magnetic resonance or nuclear spin resonance system.

In one embodiment, an exemplary computer system may be used to implement the method described herein (e.g., as a computer of the planning system). The computer system may include a display for viewing system information, and a keyboard and pointing device for data entry, screen navigation, etc. A computer mouse or other device that points to or otherwise identifies a location, action, etc., e.g., by a point and click method or some other method, are examples of a pointing device. Alternatively, a touch screen may be used in place of the keyboard and pointing device. The display, keyboard and mouse communicate with a processor via an input/output device, such as a video card and/or serial port (e.g., a USB port or the like).

A processor, such as an AMD Athlon 64®. processor or an Intel Pentium IV®. processor, combined with a memory source execute programs to perform various functions, such as data entry, numerical calculations, screen display, system setup, etc. The memory may comprise several devices, including volatile and non-volatile memory components. Accordingly, the memory may include, for example, random access memory (RAM), read-only memory (ROM), hard disks, floppy disks, optical disks (e.g., CDs and DVDs), tapes, flash devices and/or other memory components, plus associated drives, players and/or readers for the memory devices. The processor and the memory are coupled together via a local interface. The local interface may be, for example, a data bus with accompanying control bus, a network, or other subsystem.

The memory may form part of a storage medium for storing information, such as application data, screen information, programs, etc., part of which may be in the form of a database. The storage medium may be a hard drive, for example, or any other storage means that can retain data, including other magnetic and/or optical storage devices. A network interface card (NIC) allows the computer system to communicate with other devices.

A person having ordinary skill in the art of computer programming and applications of programming for computer systems would be able in view of the description provided herein to program a computer system to operate and to carry out the functions described herein. Accordingly, details as to the specific programming code have been omitted for the sake of brevity. Also, while software in the memory or in some other memory of the computer and/or server may be used to allow the system to carry out the functions and features described herein in accordance with the preferred embodiment of the invention, such functions and features also could be carried out via dedicated hardware, firmware, software, or combinations thereof, without departing from the scope of the invention.

Computer program elements of the invention may be embodied in hardware and/or in software (including firmware, resident software, micro-code, etc.). The invention may take the form of a computer program product, which can be embodied by a computer-usable or computer-readable storage medium having computer-usable or computer-readable program instructions, “code” or a “computer program” embodied in the medium for use by or in connection with the instruction execution system. In the context of this document, a computer-usable or computer-readable medium may be any medium that can contain, store, communicate, propagate, or transport the program for use by or in connection with the instruction execution system, apparatus, or device. The computer-usable or computer-readable medium may be, for example but not limited to, an electronic, magnetic, optical, electromagnetic, infrared, or semiconductor system, apparatus, device, or propagation medium such as the Internet. Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner. The computer program product and any software and hardware described herein form the various means for carrying out the functions of the invention in the example embodiments.

Although the invention has been shown and described with respect to a certain preferred embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

In particular, the invention relates to a method for detecting the exact contour of targeted treatment areas, in particular, the external contour, i.e. mapping surgical target site contours precisely, especially the outer contour thereof.

In lesion surgery, use is made of invasive or non-invasive (radiation therapy) methods, in which it is principally desirable to be aware of the precise outer contours of the lesion so as to avoid disturbing healthy tissue surrounding the lesion during surgery. It is particularly important in the case of brain surgery to precisely distinguish between healthy and diseased tissue in deciding implementation invasive or non-invasive.

Computer-assisted surgery is useful, whereby surgical target site data is mapped by means of CT or MRI tomography and a referencing method, stored as slice images for various mapping planes and made available to the surgeon via a computer system and a display monitor.

In accordance with the methods used to date the surgeon in preparing for the operation marks the lesion requiring surgery or its contour in each slice plane with the aid of a display cursor. Marking is done manually for each of the slice planes in sequence, i.e. in each of three directions standing perpendicular to the other so that from the information provided as a whole the three-dimensional configuration of the lesion can be computed. It is with this information as to the configuration and location of the lesion that computer-assisted surgery can then be undertaken, the outer contour, i.e. distinguishing healthy surrounding tissue being particularly important in this respect.

The main drawback of this conventional method of marking the contour is its relative inaccuracy, due not only to the marking being done manually but also attributable to other unavoidable factors.

Thus, on the one hand, in the images displayable on the computer the transitions between healthy and diseased tissue are often quite unclear or blurred and accordingly hardly discernible to the naked eye, while on the other, it may be that a healthy structure is just located in the surroundings of the diseased tissue which furnishes the same color or gray scale value as the diseased tissue in the computer display. It is particularly in the latter case that making a distinction between the two with the naked eye is very difficult or even impossible and there is thus the risk that marking lacking sufficient precision is to the detriment of healthy tissue in the operation on the basis of the marking data. Particularly in the brain quite natural changes in density exist in many locations which detriment or even make it impossible to detect the outer contour of lesions as described above.

It is thus the object of the present invention to provide a method for mapping surgical target site contours precisely, especially the outer contour thereof, which obviates the disadvantages of methods described above. More particularly, the intention is to make possible a precise contour, more particularly outer contour mapping of surgery target sites.

This object is achieved in accordance with the invention by a method for mapping surgical target site contours precisely, especially the outer contour thereof comprising the following steps:

producing a plane slice image through the surgery site in the region of the surgery target whereby the plane of the slice image is located substantially perpendicular to a plane of symmetry of the surgery target site;

assigning the images split by the plane of symmetry, more particularly by mirroring at the plane of symmetry; and

establishing the difference in the image contents between the assigned split images and processing the resulting information to determine the location of the contour of the surgery target site, especially the outer contour thereof in the plane of the slice image.

In other words, the problem of inaccuracies in the case of symmetrical surgery target sites, as often exists, for example, in the human brain, is solved in that the information taken from a symmetrical healthy counterpart to the diseased surgery target site is made use of in mapping the contour. When, for example, a healthy change in density (bone, denser tissue) produces in the vicinity of the lesion the same gray scale or color value in the computer image and thus permits precise mapping of the contour, the outer contour of such a non-diseased tissue structure may be precisely mapped with the aid of the image of the assigned healthy structure. Thus, when the “healthy image” is assigned to the “diseased image”, for example by overlapping or mirroring at the plane of symmetry it is accordingly possible to advantage in accordance with the invention, despite the imprecise contour information from the “diseased image”, to obtain a precise distinction between healthy and diseased tissue since it is now simple to establish that it is there where healthy tissue no longer exists, that diseased tissue must commence.

By means of the method in accordance with the invention it has now become possible for the first time to map the contour of surgery targets in such difficult sites and, in addition to this, may now also be implemented with high accuracy.

The slice images required may be produced in three planes by means of an imaging method, more particularly by CT or MRI or by PET or SPECT tomography.

In one preferred embodiment of the present invention assigning the split images is done with the aid of the computer by means of stored slice image data.

There is now the possibility of establishing the difference in the image contents by forming the difference of locally assigned color or gray scale values in the various split images. The tissue is thus segmented by subtracting the color or gray scale values, i.e. preferably by a digital computer-assisted subtraction to thus “filter out” the healthy tissue from the image.

As already indicated, the mapping data of the contour, especially the outer contour, may be stored and following sensing in several planes of the slice image may be made use of to define the complete contour, especially the outer contour of the surgery target site. Accordingly, after implementation of the method in accordance with the invention for subsequent slice images three-directionally the precise outer contour of the surgery target site is available for display three-dimensionally or in three slice planes on the computer and for use in assisting surgery.

It is to be noted in this respect that by means of the contour mapping in accordance with the invention, when implemented computer-assisted, preparing for the operation is now possible much more quickly than by conventional manual contour marking means.

More particularly the method in accordance with the invention lends itself in preparing for invasive or non-invasive brain surgery, the brain having in many areas a sufficiently symmetrical structure parted by the so-called brain centerline (brain centerplane). The difficulties as cited above in manual or visual contour definition in accordance with prior art are also to be anticipated all the more where the brain is involved since it is here that often natural changes in density exist. This is why, in a method in accordance with the invention aimed at brain surgery the brain centerline is made use for assigning the images split by the axis of symmetry, especially as the mirroring axis.

Assignment may be rendered even more precise by also precisely superimposing natural landmarks, for example bone ends or explicitly identifiable tissue contours with the aid of the computer. In all, the present invention thus provides a fast, precise means of making it possible for the first time to map the contour of surgery target site.

Although the embodiments have been described relative to various selected non-limiting examples, there are numerous variations and modifications that will be readily apparent to those skilled in the art in light of the above teachings. It is therefore to be understood that, within the scope of the claims hereto attached and supported by this specification, the embodiments may be practiced other than as specifically described.

1. A surgical planning and navigation system comprising:
a) a computer capable of displaying various informational images related to any specific voxel in a body part of a patient for clinical diagnosis and surgical navigation;
b) optional stationary MRI compatible fiducial markers attached to rigid and unaffected tissue outside the area of surgical intervention of said patient, wherein fiducial markers enable the computer to co-register the various informational images related to any specific voxel in said body part from the images acquired prior, during, and after the surgical intervention promptly without considering defects produced by the surgical intervention;
c) a probe that is connected to the computer such that the computer can navigate the probe to a specific voxel; and
d) one or more electrodes, catheters, optical fibers or other investigational or treatment devices directed via the probe to a targeted voxel(s) in the tissue, wherein the investigational or treatment devices are capable of relaying real time functional information related to the targeted voxel(s) in the present surgical planning and navigation system.

2. The surgical planning and navigation system of claim 1, wherein the targeted voxel(s) in the tissue are within a region to be subjected to surgical intervention.

3. The surgical planning and navigation system of claim 2, wherein the surgical intervention is planned for the treatment of epilepsy, tumor, stroke or other diseases which requires the guidance from medical images.

4. The surgical planning and navigation system of claim 3, wherein the fiducial markers are made of a non-paramagnetic material.

5. The surgical planning and navigation system of claim 4, wherein the non-paramagnetic material is gold.

6. The surgical planning and navigation system of claim 4, wherein the real time information related to the targeted voxel(s) is related to blood flow, oxygenation, metabolism, or other data related to physiological function of the tissue.

7. The surgical planning and navigation system of claim 6, wherein the real time information related to the targeted voxel(s) in the tissue provides guidance to an operator of the system for planning surgical intervention of the tissue.

8. The surgical planning and navigation system of claim 7, wherein during the surgical intervention of the tissue, the tissue is scanned using MRI.

9. The surgical navigation planning and system of claim 8, wherein the MRI scans of the tissue can be pre-operative, intra-operative or post-operative scans are used to pre-surgical planning and to monitor the progress the planned surgical intervention of the tissue.

10. The surgical planning and navigation system of claim 9, wherein the monitoring of the progress the planned surgical intervention of the tissue guides the operator of the system during the planned surgical intervention of the tissue.

11. The surgical planning and navigation system of claim 10, wherein the fiducial markers remain attached to the rigid and unaffected tissue outside surgical intervention of the patient throughout the planned surgical intervention of the tissue for use in pre-operative, intra-operative or post-operative MRI scan.

12. The surgical planning and navigation system of claim 11, wherein the post-operative MRI scans of the tissue are used to confirm the success of the planned surgical intervention of the tissue.

13. The surgical planning and navigation system of claim 12, wherein the fiducial markers and further scans using MRI are used in the planning of additional planned surgical intervention of the tissue.

14. The surgical planning and navigation system of claim 1, wherein the fiducial makers are utilized for prompt co-registration alignment between pre-intervention images and post-intervention images without considering the image changes resultant of the surgical intervention.

15. The surgical planning and navigation system of claim 1, wherein the patient is a pediatric patient.

16. A method of treating epilepsy, tumor, stroke or other diseases which requires the guidance from medical images in a patient using the surgical planning and navigation system of claim 1.

17. A method of ameliorating epilepsy, tumor, stroke or other diseases which requires the guidance from medical images in a patient using the surgical planning and navigation system of claim 1.

18. A method of preventing future epileptic episodes in a patient using the surgical planning and navigation system of claim 1.

19. A method of using the surgical planning and navigation system of claim 1 for the treatment, amelioration, or prevention of epileptic episodes in a patient.

20. The method of claim 19, wherein the patient is a pediatric patient of age 5 or younger.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)
